SEQUENCE LISTING (6 sequences)

SEQ ID NO: 1 - protein, 445 aa, synthetic construct
  REGION 1..445: note = The sequence is synthetized.
EVQLVESGGGLVKPGGSLRLSCAASGFTFSNYAMSWVRQAPGKGLEWVATISSGGSHTYYLDSVKGRFTISRDNSKNTLYLQMNSLRAEDTALYYCARLFTGYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 216 aa, synthetic construct
  REGION 1..216: note = The sequence is synthetized.
QIVLTQSPATLSLSPGERATMSCTASSSVSSSYLHWYQQKPGKAPKLWIYSTSNLASGVPSRFSGSGSGTDYTLTISSLQPEDFATYYCHQYYRLPPITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, synthetic construct
  REGION 1..10: note = The sequence is synthetized.
GYLGRSYKVG

SEQ ID NO: 4 - protein, 8 aa, synthetic construct
  REGION 1..8: note = The sequence is synthetized.
YVGRSYRG

SEQ ID NO: 5 - protein, 8 aa, synthetic construct
  REGION 1..8: note = The sequence is synthetized.
GLQRALEI

SEQ ID NO: 6 - protein, 7 aa, synthetic construct
  REGION 1..7: note = The sequence is synthetized.
SLGRKIQ

(19) United States
(12) Patent Application Publication
(10) Pub. No.: US 2025/0012721 A1
(43) Pub. Date: Jan. 9, 2025

(54) FLUORESCENCE DETECTION KIT FOR ACTIVATED COMPLEMENT C1s, DETECTION METHOD USING SAME, AND USE THEREOF

(71) Applicant: JIANGSU UNIVERSITY, Zhenjiang (CN)

(72) Inventors: Sheng XIA, Zhenjiang (CN); Jun YE, Zhenjiang (CN)

(73) Assignee: JIANGSU UNIVERSITY, Zhenjiang (CN)

(21) Appl. No.: 18/262,927

(22) PCT Filed: Aug. 26, 2022
(86) PCT No.: PCT/CN2022/115117

§ 371 Date: Jul. 26, 2023

(30) Foreign Application Priority Data
May 19, 2022 (TR) 202210563184.7

(51) Int. Cl. G01N 21/64; C07K 16/40; G01N 33/543; G01N 33/573; G01N 33/58

(52) U.S. Cl. CPC G01N 21/6428; C07K 16/40; G01N 33/54326; G01N 33/573; G01N 33/582; C07K 2317/33; G01N 2021/6439; G01N 2333/96433

(57) ABSTRACT

A fluorescence detection kit for the enzymatic activity of activated complement C1s, a detection method using the same, and use of the same are provided for a purpose of detecting the enzymatic activity level of the activated complement C1s in a sample. Specific capture and magnetic separation of C1s in a C1s standard or a sample to be tested are performed based on a magnetic bead precoated with a C1s recombinant antibody. After non-specific binders are removed, a fluorescence resonance energy transfer (FRET) fluorescein labeled substrate peptide for enzyme digestion by C1s is added, and a fluorescence signal in an obtained reaction system is detected with a microplate reader. Accordingly, the enzymatic activity of activated C1s is quantitatively determined by detecting changes of a fluorescence intensity.

### CROSS REFERENCE TO THE RELATED APPLICATIONS

This application is the national phase entry of International Application No. PCT/CN2022/115117, filed on Aug. 26, 2022, which is based upon and claims priority to Chinese Patent Application No. 202210563184.7, filed on May 19, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBNJIP114_Sequence_Listing.xml, created on 07/13/2023, and is 9,065 bytes in size.

### TECHNICAL FIELD

The present disclosure belongs to the field of biotechnology, and specifically relates to a fluorescence detection kit for an enzymatic activity of activated complement C1s, a detection method using the same, and use of the same.

### BACKGROUND

As a class of natural immune molecules, complements are composed of various complement molecules and their regulatory molecules, and play an important role in immune response. Under normal physiological conditions, complement molecules are mostly in an inactive state. After the immune response is activated, the complement molecules are activated to exhibit various biological activities. A complement activation pathway starting from complement molecule C1 (C1 for short) is called a classical pathway of complement activation. The C1 is a pentameric macromolecular complex (C1qC1r2C1s2) including three subunits of C1q, C1r, and C1s at a ratio of 1:2:2 under the action of Ca²⁺. The C1q has six identical immunoglobulin (Ig) binding sites. When two or more of these binding points bind to a complement-binding site of an Fc fragment of IgM or IgG in the immune complexes, the configuration of C1q changes and then activates the subunit C1r. Activated C1r further activates the C1s. Activated C1s shows an enzymatic activity, and can cleave complements C4 and C2, thereby eventually making subsequently activated complement components form a transmembrane channel (namely an attack complex) on a target cell membrane. The activation of C1s is related to the occurrence, development, and prognosis of infections, tumors, cold agglutinin disease, thrombocytopenic purpura, systemic lupus erythematosus and other diseases.

In recent years, therapeutic drugs developed targeting C1s have begun to enter clinical trials. Diseases involved include cold agglutinin disease (CAD), warm autoimmune hemolytic anemia (wAIHA), bullous pemphigoid (BP), and antibody-mediated rejection (AMR). Under normal physiological conditions, C1s exists in the state of proenzyme. Only when the C1s proenzyme is activated to become activated C1s can it exert a biological activity effect. In view of this, only the accurate detection of the enzymatic activity of activated C1s in samples to be tested can correctly reflect the complement activation of classical pathway and the functional status of activated C1s. Therefore, it is of great significance for understanding a mechanism of a disease, a state of the disease, and clinical individualized treatment of the disease by detecting the change of C1s enzymatic activity in vivo. Currently, detection methods for C1s include bidirectional diffusion assay, enzyme-linked immunosorbent assay (ELISA), and gelatin zymography. However, these methods can only detect a total protein content of C1s in clinical blood samples, and cannot effectively distinguish an activated or non-activated state of the C1s. That is, these methods cannot objectively reflect an enzymatic activity of the C1s. As a result, it is necessary to establish a sensitive and specific detection method for the enzymatic activity of activated C is, to meet clinical requirements for detection of the activated C1s in samples to be tested.

### SUMMARY

In view of this, an objective of the present disclosure is to provide a fluorescence detection kit for the enzymatic activity of activated complement C1s, and a detection method for the enzymatic activity of activated complement C1s using the same. The kit can be applied to detection of the enzymatic activity of activated complement C1s in blood and body fluids (such as pleural effusion, ascites, and cerebrospinal fluid) of human and animals, to evaluate a complement activation state in vivo. The present disclosure satisfies the demands for complement activity detection in applications such as clinical disease diagnosis, individualized treatment, prognosis assessment, and disease mechanism research.

To achieve the above objective, the present disclosure adopts the following technical solutions.

The present disclosure provides a C1s-specific recombinant antibody, where a heavy chain variable region of the C1s-specific recombinant antibody has an amino acid sequence shown in SEQ ID NO: 1, and a light chain variable region of the C1s-specific recombinant antibody has an amino acid sequence shown in SEQ ID NO: 2.

The present disclosure further provides an immunomagnetic bead, where the immunomagnetic bead is coupled with the C1s-specific recombinant antibody.

The present disclosure further provides a fluorescence detection kit for the enzymatic activity of activated complement C1s, including the immunomagnetic bead, and further including a C1s standard, a substrate peptide, a phosphate-buffered saline, and a Tris buffer.

Further, a fluorescence resonance energy transfer (FRET) fluorescent label of the substrate peptide has a complete sequence of 2Abz-GYLGRSYKVG-Lys(Dnp)D-OH, as shown in SEQ ID NO: 3.

Further, an N-terminal of the substrate peptide is ligated with Abz (a fluorophore R), and a C-terminal of the substrate peptide is ligated with Dnp (a fluorescence quencher Q).

The C1s standard is a humanized or recombinant C1s standard.

Further, the phosphate-buffered saline (PBS, pH=7.4) includes following components: 0.137 M NaCl, 0.0027 M KCl, 0.01 M Na₂HPO₄, and 0.002 M KH₂PO₄.

The Tris buffer includes following components: 0.05 M Tris, 0.15 M NaCl, and 0.2% (w/v) polyethylene glycol 8000.

The present disclosure further provides a fluorescence detection method for an enzymatic activity of activated complement C1s, including following steps:
(1) preparing a C1s recombinant antibody-coupled immunomagnetic bead;
(2) mixing a sample to be tested or a standard with the C1s recombinant antibody-coupled immunomagnetic bead to obtain a resuspended immunomagnetic bead;
(3) adding the resuspended immunomagnetic bead into a substrate peptide to conduct enzyme digestion, to obtain a supernatant;
(4) detecting a fluorescence signal in the supernatant by a microplate reader; and
(5) performing straight line fitting by taking a C1s enzymatic activity as an abscissa and a fluorescence intensity as an ordinate with a double logarithmic Log-Log mathematical model and using a least square method to plot a standard curve; and calculating the enzymatic activity of an activated C1s in the sample to be tested according to an enzymatic activity of the standard combined with a fluorescence signal of the sample to be tested.

Further, in the step (3), an N-terminal of the substrate peptide is ligated with Abz, and a C-terminal of the substrate peptide is ligated with Dnp; and the substrate peptide has a sequence of 2Abz-YVGRSYRG-Lys(Dnp)-NH2, as shown in SEQ ID NO: 4.

The present disclosure further provides use of the substrate peptide in preparation of a kit for detecting an activation state of C1s.

The present disclosure further provides use of the C1s-specific recombinant antibody in preparation of a kit for detecting an activation state of C1s.

The present disclosure further provides use of the immunomagnetic bead in preparation of a kit for detecting an activation state of C1s.

Compared with the prior art, the present disclosure achieves following advantages.

In the present disclosure, for a purpose of detecting the enzymatic activity level of activated C1s in a sample to be tested, specific capture and magnetic separation of C1s in a C1s standard or a sample to be tested are performed based on an immunomagnetic bead precoated with a C1s recombinant antibody. After removing non-specific binders, a fluorescein labeled substrate peptide for enzyme digestion by C1s is added, and a fluorescence signal in an obtained reaction system is detected with a microplate reader. Accordingly, the activity of activated C1s enzyme in the sample to be tested is quantitatively determined by detecting changes of a fluorescence intensity. The kit has high sensitivity, strong specificity, and small error, and the detection method has simple and convenient operations. The kit can detect the enzymatic activity of C1s in blood and body fluids of human or animals, so as to accurately evaluate a classic state of complement activation in vivo.

### BRIEF DESCRIPTION OF THE DRAWINGS

FIG. 1 shows a method and a principle of a fluorescence detection kit for the enzymatic activity of activated C1s.

FIG. 2 shows an electropherogram of sodium dodecyl sulfate-polyacrylamide gel electrophoresis (SDS-PAGE) of a C1s recombinant antibody.

FIGS. 3A-3B show chromatogram results of size exclusion-high performance liquid chromatography (SEC-HPLC) of the C1s recombinant antibody.

FIG. 4 shows a principle of specificity detection of the C1s recombinant antibody.

FIG. 5 shows comparison of a binding specificity of the C1s recombinant antibody and a detection antigen.

FIGS. 6A-6B show effects of a C1s standard on a substrate peptide 3 of different concentrations; where FIG. 6A is enzymatic reaction kinetics of an activated C1s standard on the substrate peptide 3; and FIG. 6B is a correlation between a concentration of the substrate peptide 3 and an enzyme reaction speed.

FIG. 7 shows a standard curve illustrating enzymatic activities and fluorescence intensities of different C1s standards.

### DETAILED DESCRIPTION OF THE EMBODIMENTS

The specific examples of the present disclosure will be described in detail below with reference to the accompanying drawings and examples. The following examples are only used for describing the technical solutions of the present disclosure more clearly, and are not intended to limit the protection scope of the present disclosure. The experimental methods which are not specified with specific conditions in the following examples are performed according to the conventional methods and conditions in the art or according to the product instructions. The reagents and raw materials for which specific components are not indicated in the following examples are all commercially available.

In the present disclosure, the serum samples are from Taizhou People's Hospital. The interference check A kit is available from Sysmex Corporation (Shanghai) Co., Ltd. The reducing gel and non-reducing gel of SDS-PAGE are both from BBI Life Science Co., Ltd. The coupling agent is 1-ethyl-3-[3-dimethylaminopropyl]carbodiimide hydrochloride (EDC) from Diamond. The magnetic bead is from Merck Pharmaceuticals (Jiangsu) Co., Ltd. The Cir protein, double-stranded Cir protein, unactivated C1s protein, and double-stranded activated C1s protein are all from Complement Technology. Both MASP1 and MASP2 are from Abnova.

The present disclosure provides a C1s-specific recombinant antibody and a FRET (Abz/Dnp dye combination)-labeled substrate peptide, and provides a fluorescence detection kit for the enzymatic activity of activated complement C1s, a detection method, and use based on same. The recombinant anti-C1s antibody is coated on magnetic beads to prepare immunomagnetic beads; after washing and removing a supernatant by magnetic suction, a C1s standard or a sample to be tested is added; the anti-C1s antibody bound to the immunomagnetic beads can capture C1s in a liquid phase. After washing and magnetic separation are performed to remove other unbound components, a substrate peptide is added. Activated C1s is an important enzymatic activity-having molecule after the initiation of complement activation in a classical pathway. The activated C1s in a complement system shows a serine protease activity and can specifically cleave complements C4 and C2 proteins. The cleavage sites of the C4 and C2 proteins are selected as a central point of an amino acid sequence of the substrate peptide for enzyme digestion, and several amino acids are selected and added to left and right sides of the central point of the amino acid sequence, to prepare a basic substrate peptide for enzyme digestion. Then, Abz (fluorophore R) and Dnp (fluorescence quencher Q) are ligated to the left and right sides of this peptide, respectively, so as to obtain an enzyme digestion-targeted fluorescent substrate peptide. When there is no active C1s in a system to be tested, the substrate peptide remains intact, and the fluorophore R and the fluorescence quencher Q labeled on the substrate peptide have an interval distance of 10 Å to 100 Å; at this moment, the fluorescence quencher Q can quench fluorescence emitted by the fluorophore R, such that no fluorescence signal can be detected in the system to be tested. When there is active C1s in the system to be tested, the substrate peptide is digested by the active C1s. After that, the fluorescence quencher Q and the fluorophore R on the substrate peptide are located on different enzyme digestion fragments, respectively, and the fluorophore R is no longer quenched by the fluorescence quencher Q. The fluorescence R can emit a detectable fluorescence signal, and has a fluorescence intensity that is related to a level of the enzymatic activity of activated C1s.

Therefore, the enzymatic activity of the activated C1s in the sample to be tested can be quantitatively determined by detecting the changes of the fluorescence intensity. Meanwhile, other proteins (such as MASP2) in serum and plasma may interfere with the substrate peptide for enzyme digestion. In the present disclosure, a C1s recombinant antibody-coupled magnetic bead (immunomagnetic bead) is used to specifically capture C1s, other non-conjugated interfering substances are eluted by magnetic suction, and then the substrate peptide for enzyme digestion that specifically activate C1s is added. The enzymatic activity of activated C1s is calculated by detecting the changes of fluorescence intensity in the substrate peptide for enzyme digestion by activated C1s.

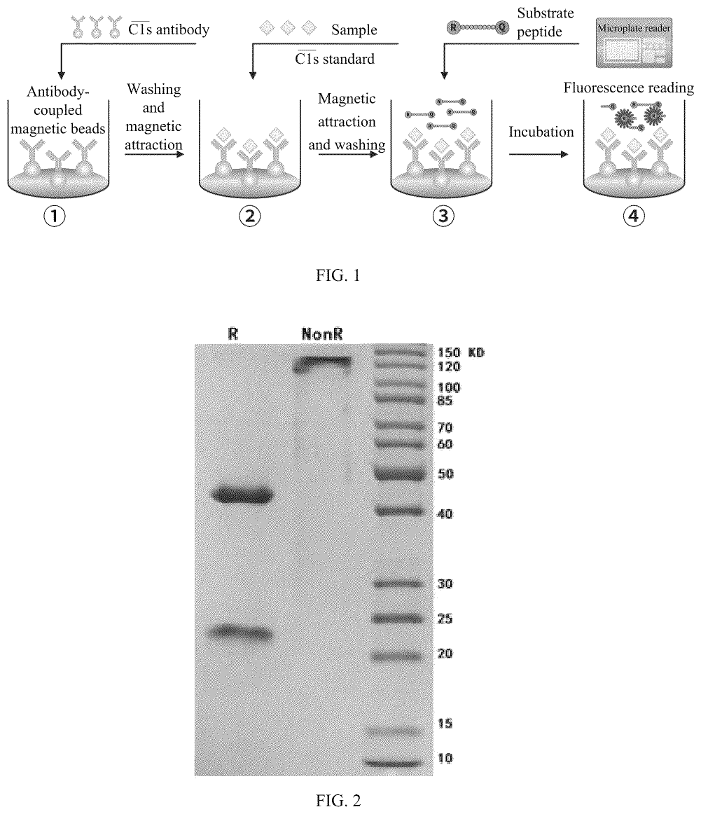

FIG. 1 shows a method and a principle of the fluorescence detection of the enzymatic activity of activated C1s. As shown in FIG. 1, specific capture and magnetic separation of C1s in a C1s standard or a sample to be tested are performed based on an immunomagnetic bead. After removing non-specific binders, a FRET fluorescein labeled substrate peptide for enzyme digestion by C1s is added to perform enzyme digestion, and then a fluorescence signal in an obtained reaction system is detected with a microplate reader.

### Example 1: Recombinant Expression and Identification of C1s Antibody

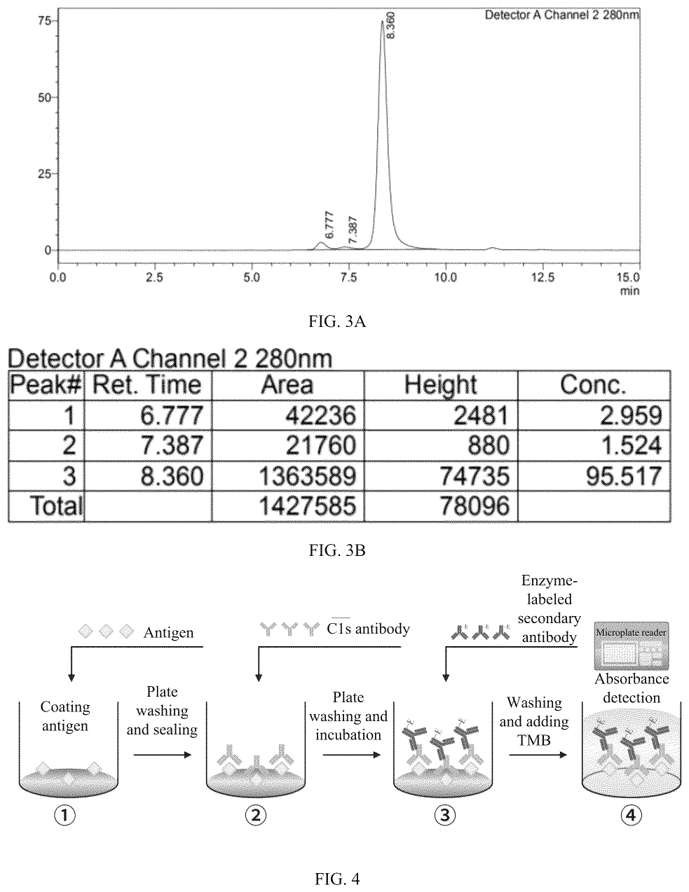

A C1s recombinant antibody had a heavy chain amino acid sequence shown in SEQ ID NO: 1, namely: EVQLVESGGGLVKPGGSLRLSCAASGFTFSNYAMSWVRQAPGKGLEWVATISSGGSHTY YLDSVKGRFTISRDNSKNTLYLQMNSLRAEDTALYYCARLFTGYAMDYWGQGTLVTVS SASTKGPSVFPLAPCSRSTSESTAALGCLVKDYFPEPVTVSWNSGALTSGVHTFPAVLQSS GLYSLSSVVTVPSSSLGTKTYTCNVDHKPSNTKVDKRVESKYGPPCPPCPAPEFEGGPSV FLFPPKPKDTLMISRTPEVTCVVVDVSQEDPEVQFNWYVDGVEVHNAKTKPREEQFNST YRVVSVLTVLHQDWLNGKEYKCKVSNKGLPSSIEKTISKAKGQPREPQVYTLPPSQEEM TKNQVSLTCLVKGFYPSDIAVEWESNGQPENNYKTTPPVLDSDGSFFLYSRLTVDKSRWQ EGNVFSCSVMHEALHNHYTQKSLSLSLGK. The C1s recombinant antibody had a light chain amino acid sequence shown in SEQ ID NO: 2, namely: QIVLTQSPATLSLSPGERATMSCTASSSVSSSYLHWYQQKPGKAPKLWIYSTSNLASGVPS RFSGSGSGTDYTLTISSLQPEDFATYYCHQYYRLPPITFGQGTKLEIKRTVAAPSVFIFPPSD EQLKSGTASVVCLLNNFYPREAKVQWKVDNALQSGNSQESVTEQDSKDSTYSLSSTLTL SKADYEKHKVYACEVTHQGLSSPVTKSFNRGEC. Taizhou Biointron Co., Ltd. was entrusted to recombine and express a C1s antibody using an HEK293T system. The obtained C1s recombinant antibody was analyzed and identified by SDS-PAGE and SEC-HPLC, respectively. FIG. 2 shows an electropherogram of SDS-PAGE of the C1s recombinant antibody. As shown in FIG. 2, the C1s recombinant antibody could be detected to have two bands on the SDS-PAGE reducing gel, with molecular weights at 49 KD and 23 KD, respectively; however, this antibody showed only one 144 KD band on the SDS-PAGE non-reducing gel. FIG. 3A shows a chromatogram of SEC-HPLC of the C1s recombinant antibody. As shown in FIG. 3B, SEC-HPLC results indicated that the C1s recombinant antibody had a purity of 95.5%.

### Example 2: Analysis of Antigen-Binding Specificity of C1s Recombinant Antibody

FIG. 4 shows a principle of specificity detection of the C1s recombinant antibody. In this example, the antigen-binding specificity of the C1s recombinant antibody was analyzed by an indirect ELISA method according to the principle of the specificity detection of the C1s recombinant antibody shown in FIG. 4. An analysis method included the following steps:

200 μL (1 μg/ml) of C1s antigen (single-stranded unactivated C1s protein, double-stranded activated C1s protein, or C1r protein) was added respectively to wells of a microtiter plate, and the microtiter plate was gently shaken to make the antigen cover a well bottom of the microtiter plate. Incubation was performed overnight at 4° C. After washing the plate 3 times with a warm PBS, 200 μL of a blocking solution (10 mM PBS, 1% BSA, 0.1% casein) was added to perform blocking. Incubation was next performed at 37° C. for 1 h. The plate was washed 5 times with PBS. 100 μL of the C1s recombinant antibody was added in each well (an initial concentration of a first well was 10 μg/mL, then diluted down to 7 wells according to a ratio of ¼ gradient to set up duplicate wells), incubation was performed at 37° C. for 1 h. The liquid in the microtiter plate was spin-dried, and the plate was washed with 200 μL of warm PBS 5 times. 100 μL of a diluted secondary antibody (Goat anti-Human IgG-HRP) was added to each well of the microtiter plate. Incubation was performed for 30 min at 37° C. The liquid in the microtiter plate was spin-dried, and the plate was washed with 200 μL of warm PBS 5 times. 100 μL of a chromogenic solution (TMB) was added to each well for a chromogenic reaction, and after incubation at 37° C. for 10 min, 50 μL of a stop solution (0.25 M HCl) was finally added to terminate the chromogenic reaction. An optical density (OD) was read at 450 nm on the microplate reader.

The binding status of the C1s recombinant antibody to the single-stranded unactivated C1s protein, double-stranded activated C1s protein, and C1r protein was separately analyzed. FIG. 5 shows comparison of a binding specificity of the C1s recombinant antibody and various C1s antigens. As shown in FIG. 5, the C1s recombinant antibody had a strong binding ability to the double-stranded activated C1s protein, a weak binding ability to the single-stranded unactivated C1s protein, and no binding ability to the Cir protein. It was seen that the C1s recombinant antibody showed desirable antigen-binding specificity to activated C1s proteins, and could specifically recognize activated C1s, thereby detecting the enzymatic activity of C1s.

### Example 3: Screening of Substrate Peptide

The activated C1s in a complement system has a serine protease activity and can specifically cleave complement C4 and C2 proteins. In the present disclosure, the cleavage sites of the C4 and C2 proteins were selected as central points of an amino acid sequence of the substrate peptide for activated C1s digestion, and several amino acids were selected and added to left and right sides of the central point of the amino acid sequence, to prepare a basic substrate peptide for enzyme digestion of activated C1s. Then, Abz (fluorophore R) and Dnp (fluorescence quencher Q) were ligated to the left and right sides of this peptide, respectively, so as to obtain an enzyme digestion-targeted fluorescent substrate peptide. Three pre-designed substrate peptides were all synthesized by Shanghai Science Peptide Biotechnology Co., Ltd. The three substrate peptides had the following sequences:
substrate peptide 1: 2Abz-GLQRALEI-Lys(Dnp)-NH2, as shown in SEQ ID NO: 5;
substrate peptide 2: 2Abz-SLGRKIQ-Lys(Dnp)-NH2, as shown in SEQ ID NO: 6; and
substrate peptide 3: 2Abz-GYLGRSYKVG-Lys(Dnp)D-OH, as shown in SEQ ID NO: 3.

The Km and Kcat values of the three substrate peptides digested by the activated C1s were measured, respectively, and an average was obtained from three repetitions. The measurement steps included:
(1) 40 μL of a substrate peptide standard was added to a first well. The substrate peptide solutions diluted with a Tris buffer to different final concentrations (245.7 μM, 122.9 μM, 61.4 μM, 30.7 μM, 7.7 μM, and 0 μM) were added in other wells at a same volume.
(2) 2.5 μL (1 mg/mL) of double-stranded activated C1s was added to each well, followed by supplementing with the Tris buffer to 100 μL.
(3) The detection was performed with a microplate reader at 360/40 excitation light wave and 460/40 emission light wave, once every 5 min for a total of 2 h, and the changes of fluorescence signal were recorded.
(4) Graphpad prism 5.0 was used for data processing to obtain Vmax and Km values. According to a formula Kcat=Vmax/molecular weight, the Km and Kcat values were calculated, and the peptide with a maximum Kcat/Km value was regarded as a candidate substrate peptide. Table 1 shows Kcat/Km values of the activated C1s on different substrate peptides.

TABLE 1
Comparison of Kcat/Km of activated C1s on different substrate peptides

| Substrates | Kcat/Km |
|---|---|
| Substrate peptide 1 | 1.67 ± 0.89 |
| Substrate peptide 2 | 1.45 ± 0.61 |
| Substrate peptide 3 | 15.38 ± 2.69 |

The results are shown in Table 1, substrate peptides 1 vs 3: t=12.461, p<0.001; substrate peptides 2 vs 3: t=12.664, p<0.001. The substrate peptide 3 showed the best effect, and a Kcat/Km value was 15.38, such that the substrate peptide 3 was selected as a substrate peptide for activated C1s.

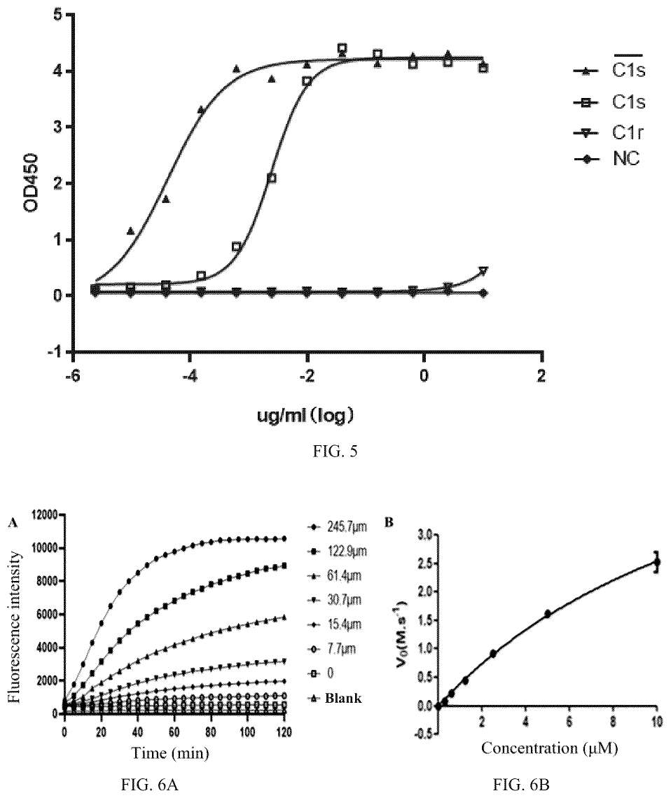

When there was no active C1s in a system to be tested, the substrate peptide had integrity, and the fluorophore R and the fluorescence quencher Q labeled on the substrate peptide had an interval distance of 10 Å to 100 Å; at this moment, the fluorescence quencher Q could quench fluorescence emitted by the fluorophore R, such that no fluorescence signal could be detected in the system to be tested. When there was active C1s in the system to be tested, the substrate peptide was digested by the active C1s. After that, the fluorescence quencher Q and the fluorophore R on the substrate peptide were located on different enzyme digestion fragments, respectively, and the fluorophore R was no longer quenched by the fluorescence quencher Q. The fluorophore R could emit a detectable fluorescence signal, and had a fluorescence intensity that was related to an amount of the activated C1s. FIGS. 6A-6B show effects of a C1s standard on a substrate peptide 3 of different concentrations; where FIG. 6A shows enzymatic reaction kinetics of an activated C1s standard on the substrate peptide 3; and FIG. 6B shows a correlation between a concentration of the substrate peptide 3 and an enzyme reaction speed. As shown in FIGS. 6A-6B, as the time changed, the fluorescence intensity of activated C1s on substrate peptide 3 of different concentrations was continuously enhanced and changed; moreover, there was a better resolution between the reactions of substrate peptides with different concentrations.

### Example 4: Detection of Enzymatic Activity of C1s Standard

In the present disclosure, the enzymatic activity of activated C1s was detected in a sample to be tested. Therefore, it was necessary to determine the enzymatic activity of a C1s standard used in the experiment and to prepare a standard curve for later C1s enzymatic activity. The enzymatic activity of the activated C1s was detected by an N-carboxybenzyloxythiobenzyl ester/5,5′-dithiobis(2-nitrobenzoic acid) colorimetric method. The method specifically included the steps as follows:
(1) Materials: 1) Analysis buffer: 50 mM Tris, 250 mM NaCl, pH=8.0;
2) substrate: N-carboxybenzyloxythiobenzyl ester (ZKSBzl), dissolved in 10 mM dimethyl sulfoxide; and
3) 5,5′-dithiobis(2-nitrobenzoic acid) (DTNB), dissolved in 10 mM dimethyl sulfoxide.
(2) Detection of C1s activity units:
1. The C1s standard was diluted to 0.2 μg/mL with an assay buffer.
2. The DNTB was added to the assay buffer at a final concentration of 200 μM, and a substrate was added to the assay buffer at a final concentration of 1,000 μM.
3. 50 μL of 0.2 ng/μL C1s was added into a plate.
4. 50 μL of a mixture including 1,000 μM substrate/200 μM DTNB was added to the wells to start a reaction.
5. An absorbance at 5 min was read at 405 nm with a microplate reader.
(3) Calculation of activated C1s:

$$Specific Activity ( p mol / min / μg ) = \frac{Adjusted {V}_{max} * ( OD / min ) \times well volume ( L ) \times {10}^{12} p mol / mol}{ext . {coeff}^{**} ( {M}^{- 1} {cm}^{- 1} ) \times path corr {.}^{** *} ( cm ) \times amount of enzyme ( μg )}$$

*Adjusted for Substrate Blank; **Using the extinction coefficient 13260 M1cm1; ***Using the path correction 0.320 cm.
(4) Test results: 1 μg/mL C1s standard showed an enzymatic activity of 0.91 pmol/min/μg. In subsequent experiments, the enzymatic activity of the C1s standard was defined in this way.

### Example 5: Methodological Establishment and Optimal Reaction Time of Fluorescence Detection of Activated C1s

1. Preparation of a buffer: 1) Binding Buffer: 0.1 M morpholinoethanesulfonic acid (MES, pH=5.0);
2) Washing Buffer: TBS containing 0.05% Tween 20;
3) Blocking Buffer: containing 0.05 M Tris, 1.0% BSA, and 0.05% Tween 20;
4) Desired Buffer: Tris buffer containing 0.05% Tween 20 and BSA; and
5) Tris Buffer: 0.05 M Tris, 0.15 M NaCl, and 0.2% (w/v) polyethylene glycol 8000.
2. Preparation of immunomagnetic beads (C1s recombinant antibody-coupled magnetic beads)
(1) 10 mg of magnetic beads (Merck carboxyl magnetic beads, with a particle size of 1 μm) were washed 3 times with 1 mL of Binding Buffer, and vortexed to mix evenly.
(2) 20 μg of the C1s recombinant antibody obtained in Example 1 was added into every 1 mg of the magnetic beads, mixed well, and vortexed at a room temperature (25° C.) for 15 min.
(3) 50 μg of a coupling agent was added into every 1 mg of the magnetic beads, mixed well, and vortexed at a room temperature for 1 h.
(4) 1 mL of Binding Buffer was added and vortexed at a room temperature for 3 h.
(5) The magnetic beads were washed 4 times with 0.5 mL of Washing Buffer.
(6) The magnetic beads were resuspended with 1 mL of Desired Buffer to obtain C1s recombinant antibody-coupled magnetic beads with a concentration of 5 mg/mL, and stored at 4° C. until later use.
3. Detection of the enzymatic activity of activated C1s
(1) 60 μL of the C1s recombinant antibody-coupled magnetic beads were stirred evenly, subjected to magnetic suction to remove a supernatant, added with 200 μL of PBS to mix well, subjected to the magnetic suction to remove a supernatant, and washed repeatedly 2 times.
(2) 10 μL of a sample to be tested or activated C1s standards with different concentrations were diluted to 100 μL with PBS, and mixed vertically with the C1s recombinant antibody-coupled magnetic beads at a room temperature for 10 min. The magnetic suction was performed to discard a supernatant. According to the enzymatic activity of the C1s standard determined in Example 4, the enzymatic activity conversion of the C1s standards with different concentrations is shown in Table 2.

TABLE 2
Table 2 Enzymatic activity of C1s standards with different concentrations (μg/mL)

| | Concen- | Concen- | Concen- | Concen- | Concen- | Concen- | Concen- |
|---|---|---|---|---|---|---|---|
| | tration 1 | tration 2 | tration 3 | tration 4 | tration 5 | tration 6 | tration 7 |
| μg/mL | 200 | 100 | 50 | 25 | 12.5 | 6.25 | 0 |
| pmol/min/μg | 182 | 91 | 45.5 | 22.8 | 11.4 | 5.7 | 0 |

(3) The magnetic beads were repeatedly washed 3 times with 100 μL of PBS.
(4) The magnetic beads were resuspended with 90 μL of Tris buffer.
(5) 10 μL of substrate peptide 3 was added at a concentration of 1 μg/mL.
(6) The detection was performed with a microplate reader at 360/40 excitation light wave and 460/40 emission light wave, with a best reaction time of 1 h as a detection point, and the fluorescence signals were measured and recorded. The straight line fitting was performed by taking a C1s enzymatic activity as an abscissa and a fluorescence signal as an ordinate with a double logarithmic Log-Log mathematical model and using a least square method to plot a standard curve.

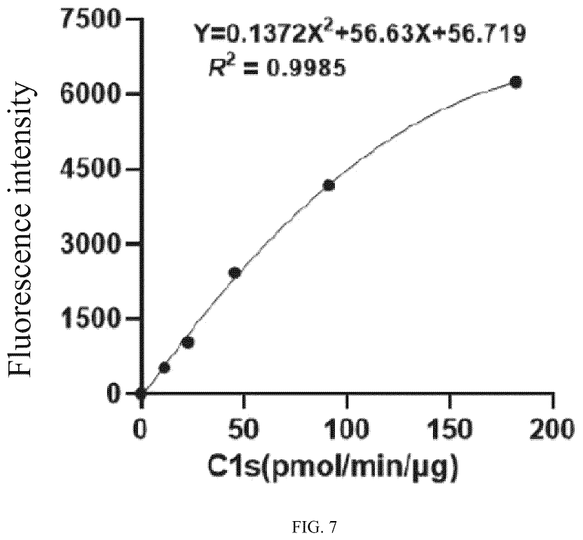

FIG. 7 shows a standard curve illustrating different enzymatic activities and fluorescence intensities of C1s standards. As shown in FIG. 7, when the detection time was 60 min, the standard curve showed R²=0.9985, and there was no hook effect in the range of a dose-response curve.

### Example 6: Sensitivity Experiment

In this example, a sensitivity experiment was performed on the fluorescence detection method for the enzymatic activity of active C1s. Referring to the steps in Example 5, a zero-value calibrator (A) was repeatedly determined 20 times, and a mean (M) and a standard deviation (SD) of VA were calculated. A concentration obtained by adding the mean of VA to twice the standard deviation (M+2SD), subtracting a background value and substituting an obtained value into the dose-response curve is the sensitivity of activated C1s determined by this method. By calculation, the C1s enzymatic activity had a minimum limit of detection at 1.64 pmol/min/μg.

### Example 7: Recovery Rate Experiment

In this example, a recovery rate experiment of the fluorescence detection method for the active C1s was performed.

3 serum samples from Taizhou People's Hospital were selected. Taking the substrate peptide 3 as a reaction substrate, the enzymatic activity of activated C1s in the serum samples was measured with reference to the steps of Example 5. It was determined that the activated C1s in the serum samples had activities of 33.49 pmol/min/μg, 33.82 pmol/min/μg, and 4.19 pmol/min/μg, respectively. 45.50 pmol/min/μg, 45.50 pmol/min/μg, and 91 pmol/min/μg of the activated C1s (volume ratio 1:1) were added to the serum samples, and the detection was performed secondly according to the method in Example 5. Each sample was measured 3 times in parallel, and measured values were recorded and calculated. The recovery rate was calculated according to the following formula: Recovery rate=(content after adding standard−sample content)/content of standard added×100%.

The results showed that the detection method had recovery rates of 91.21%, 102.75%, and 90.35%, respectively. It was seen that the recovery rate was at 90% to 110%.

### Example 8: Precision Experiment

Referring to the EP-5 document of the National Committee for Clinical Laboratory Standardization (NCCLS), from the serum samples collected in Taizhou People's Hospital, 3 samples of high, medium, and low values were selected for detection to evaluate the precision of the detection method. Table 2 shows the experimental data.

TABLE 3
Intra-assay and inter-assay precision tests of
fluorescence detection method for activated C1s

| | Intra-assay | | | Inter-assay | | |
|---|---|---|---|---|---|---|
| | (n = 10) | | | (n = 10) | | |
| | M | SD | CV (%) | M | SD | CV (%) |
| Low value | 5.34 | 0.47 | 8.80 | 5.07 | 0.48 | 9.47 |
| Medium value | 27.06 | 1.65 | 6.10 | 28.39 | 2.66 | 9.37 |
| High value | 77.80 | 5.20 | 6.68 | 75.17 | 6.37 | 8.47 |

Note:
M—mean; SD—standard deviation; CV—coefficient of variation.

As shown in Table 3, the intra-assay CV values of the high-, medium-, and low-value 3 samples were 6.68%, 6.10%, and 8.80%, respectively. The inter-assay CV values were 8.47%, 9.37%, and 9.47%, respectively. The intra-assay and inter-assay CVs were both less than 10.0%. It was seen that the detection method provided by the present disclosure had desirable precision and met the requirements of the EP-5 document of NCCLS.

### Example 9: Cross Reaction Experiment

In this example, the specificity of the activated C1s was tested. The interfering antigenic substances (complement analogues) that might exist in serum were detected, such as double-stranded C1r, MASP1, and MASP2. The double-stranded C1r, MASP1, and MASP2 were separately diluted with PBS, and the diluted concentrations were 0.66 mg/mL, 5 mg/mL, and 5 mg/mL, successively. The complement analogues were detected based on the steps of Example 5. A cross reaction rate of various substances was calculated. Cross reaction rate=measured value/theoretical value×100%. Table 4 shows data of the cross reaction experiment of the fluorescence detection method for the activated C1s. As shown in Table 4, the cross reaction rates of double-stranded C1r, MASP1, and MASP2 were 0.3%, 0.3%, and 0.04%, respectively. The cross reaction rates were all less than 0.5%. It was seen that this immunoassay had less cross reaction rates with double-stranded C1r, MASP1, and MASP2, could specifically recognize activated C1s in serum samples, and showed a high detection specificity.

TABLE 4
Cross reaction test of fluorescence detection method for activated C1s

| | | Actual | Cross reaction |
|---|---|---|---|
| Interferent | Measured concentration | concentration | rate (%) |
| Double- | 0.91 pmol/min/μg (1 μg/ | 660 μg/mL | 0.15 |
| stranded C1r | mL) | | |
| MASP1 | 1.82 pmol/min/μg (2 μg/ | 5000 μg/mL | 0.04 |
| | mL) | | |
| MASP2 | 1.64 pmol/min/μg (1.8 μg/ | 5000 μg/mL | 0.04 |
| | mL) | | |

### Example 10: Anti-Interference Experiment

The possibility that bilirubin, chyle, and hemoglobin in clinical samples cause interference in the experiment was taken into account. Referring to the interference check A kit, high-, medium-, and low-concentration standards were added with different concentrations of bilirubin (with final concentrations of 0 mg/mL, 0.06 mg/mL, 0.14 mg/mL, and 0.20 mg/mL, respectively), chyle (with final concentrations of 0 FTU, 600 FTU, 1,400 FTU, and 2,000 FTU, respectively), and a hemoglobin solution (with final concentrations of 0 mg/mL, 1.5 mg/mL, 3.5 mg/mL, and 5 mg/mL, respectively). The measured high, medium and low average concentrations containing different interfering substances were subtracted from the corresponding average concentrations without interfering substances, and a difference and a percentage of the difference in an average concentration of a blank control without interfering substances were calculated. A percentage less than 5% represented that the corresponding concentration of the interfering substance did not interfere with the detection, and a percentage greater than 5% represented that there was interference with the detection.

TABLE 5
Bilirubin interference experiment of fluorescence detection
method for activated C1s (n = 4)

| | 0 mg/mL | 0.06 mg/mL | 0.14 mg/mL | 0.20 mg/mL |
|---|---|---|---|---|
| Low value | 5.50 ± 0.32 | 5.85 ± 0.32 | 5.70 ± 0.25 | 5.61 ± 0.42 |
| (pmol/min/μg) | | | | |
| Medium value | 29.78 ± 2.82 | 31.04 ± 2.32 | 30.80 ± 1.31 | 26.93 ± 2.09 |
| (pmol/min/μg) | | | | |
| High value | 56.59 ± 2.65 | 57.01 ± 4.59 | 55.50 ± 4.50 | 55.86 ± 3.15 |
| (pmol/min/μg) | | | | |

TABLE 6
Chyle interference experiment of fluorescence detection method
for activated C1s (n = 4)

| | 0 FTU | 600 FTU | 1400 FTU | 2000 FTU |
|---|---|---|---|---|
| Low value | 5.78 ± 0.57 | 5.87 ± 0.30 | 5.68 ± 0.68 | 5.53 ± 0.95 |
| (pmol/min/μg) | | | | |
| Medium value | 32.20 ± 2.22 | 33.09 ± 3.26 | 29.63 ± 1.58 | 33.94 ± 2.81 |
| (pmol/min/μg) | | | | |
| High value | 56.57 ± 2.10 | 55.96 ± 2.30 | 54.20 ± 5.40 | 55.36 ± 5.51 |
| (pmol/min/μg) | | | | |

Note:
FTU represented formazin turbidity

TABLE 7
Hemoglobin interference experiment of fluorescence detection
method for activated C1s (n = 4)

| | 0 mg/mL | 1.5 mg/mL | 3.5 mg/mL | 5 mg/mL |
|---|---|---|---|---|
| Low value | 6.24 ± 0.43 | 6.00 ± 0.59 | 6.2 ± 0.56 | 6.37 ± 0.51 |
| (pmol/min/μg) | | | | |
| Medium value | 30.66 ± 2.90 | 29.47 ± 1.15 | 28.16 ± 2.68 | 28.23 ± 1.45 |
| (pmol/min/μg) | | | | |
| High value | 58.02 ± 4.10 | 60.57 ± 3.66 | 56.23 ± 4.58 | 59.50 ± 5.13 |
| (pmol/min/μg) | | | | |

As shown in Tables 5, 6, and 7, in the presence of high concentrations of bilirubin (0.20 mg/mL), high concentrations of chyle (2,000 FTU), and high concentrations of hemoglobin (4 mg/mL), the test results were all within an allowable range of measurement deviation (the results were based on the CV being less than 10%). The results showed that the detection method provided by the present disclosure was not interfered by bilirubin (0.20 mg/mL), chyle mixture (2,000 FTU), and hemoglobin (5 mg/mL). The data measured by the above anti-interference experiments all met the requirements.

The specific examples of the present disclosure have been described in detail above, but they are only examples, and the present disclosure is not limited to the specific examples described above. For those skilled in the art, any equivalent modifications and substitutions to the present disclosure are also within the scope of the present disclosure. Therefore, equivalent changes and modifications made without departing from the spirit and scope of the present disclosure shall fall within the scope of the present disclosure.

What is claimed is:

1. A C1s-specific recombinant antibody, wherein a heavy chain variable region of the C1s-specific recombinant antibody has the amino acid sequence shown in SEO ID NO: 1, and a light chain variable region of the C1s-specific recombinant antibody has the amino acid sequence shown in SEO ID NO: 2.

2. An immunomagnetic bead, wherein the immunomagnetic bead is coupled with the C1s-specific recombinant antibody according to claim 1.

3. A fluorescence detection kit for an enzymatic activity of an activated complement C1s, comprising the immunomagnetic bead according to claim 2, and further comprising a C1s standard, a substrate peptide, a phosphate-buffered saline, and a Tris buffer.

4. The fluorescence detection kit for the enzymatic activity of the activated complement C1s according to claim 3, wherein a fluorescence resonance energy transfer (FRET) fluorescent label of the substrate peptide has the sequence of 2Abz-GYLGRSYKVG-Lys(Dnp)D-OH, as shown in SEO ID NO: 3.

5. The fluorescence detection kit for the enzymatic activity of the activated complement C1s according to claim 3, wherein an N-terminal of the substrate peptide is ligated with a fluorophore R, and a C-terminal of the substrate peptide is ligated with a fluorescence quencher Q; and the C1s standard is a humanized or recombinant C1s standard.

6. The fluorescence detection kit for the enzymatic activity of the activated complement C1s according to claim 3, wherein the phosphate-buffered saline comprises the following components: 0.137 M NaCl, 0.0027 M KCl, 0.01 M Na₂HPO₄, and 0.002 M KH₂PO₄; and the Tris buffer comprises the following components: 0.05 M Tris, 0.15 M NaCl, and 0.2% polyethylene glycol 8000.

7. A fluorescence detection method for an enzymatic activity of an activated complement C1s, comprising the following steps:
(1) preparing a C1s recombinant antibody-coupled immunomagnetic bead;
(2) mixing a sample to be tested or a standard with the C1s recombinant antibody-coupled immunomagnetic bead to obtain a resuspended immunomagnetic bead;
(3) adding the resuspended immunomagnetic bead into a substrate peptide with the sequence of 2Abz-GYLGRSYKVG-Lys(Dnp)D-OH, as shown in SEO ID NO: 3, to conduct an enzyme digestion- to obtain a supernatant;
(4) detecting a fluorescence signal in the supernatant by a microplate reader; and
(5) performing a straight line fitting by taking a C1s enzymatic activity as an abscissa and a fluorescence intensity as an ordinate with a double logarithmic Log-Log mathematical model and using a least square method to plot a standard curve; and calculating an activity of an activated C1s enzyme in the sample to be tested according to an enzymatic activity of the standard combined with a fluorescence signal of the sample to be tested.

8. A method of using the C1s-specific recombinant antibody according to claim 1 in a preparation of a kit for detecting an activation state of C1s.

9. A method of using the immunomagnetic bead according to claim 2 in a preparation of a kit for detecting an activation state of C1s.

10. A method of using the substrate peptide according to claim 7 in a preparation of a kit for detecting an activation state of C1s.
